(19) United States
(12) Patent Application Publication
(10) Pub. No.: US 2025/0251394 A1
(43) Pub. Date: Aug. 7, 2025

(54) APTAMER-BASED ELECTROCHEMICAL DRUG DETECTION ASSAY

(71) Applicant: CONCORDIA UNIVERSITY, MONTREAL (CA)

(72) Inventors: JULIEN OUELLET, MONTREAL (CA); STEVE CHAO-CHUNG SHIH, MONTREAL (CA); LASZLO KEKEDY-NAGY, MONTREAL (CA); JAMES MCALISTER PERRY, OUTREMONT (CA); ORIOL YMBERN LLORENS, COLERAINE (IE)

(21) Appl. No.: 18/856,119

(22) PCT Filed: Apr. 12, 2023
(86) PCT No.: PCT/CA2023/050500

§ 371 Date: Oct. 11, 2024

Related U.S. Application Data
(60) Provisional application No. 63/330,129, filed on Apr. 12, 2022.

(51) Int. Cl. G01N 33/543; C12N 15/115; G01N 33/53; G01N 33/94

(52) U.S. Cl. CPC G01N 33/5438; C12N 15/115; G01N 33/5308; G01N 33/948; C12N 2310/16

(57) ABSTRACT

Aptamers configured to specifically bind to tetrahydrocannabinol and cannabidiol are disclosed, and biosensing methods and biosensor devices are described in which such aptamers are employed for detection of tetrahydrocannabinol and/or cannabidiol, with a limit of detection in the nanomolar range. In some example implementations, aptamer-based electrochemical biosensors are disclosed for sensitive and rapid detection of tetrahydrocannabinol and/or cannabidiol. Examples of microfluidic biosensors are disclosed that may be utilized in point-of-care settings.

### RELATED APPLICATIONS

This application claims priority to U.S. Provisional Patent Application No. 63/330,129, titled “APTAMER-BASED ELECTROCHEMICAL DRUG DETECTION ASSAY” and filed on Apr. 12, 2022, which is incorporated herein by reference in its entirety.

### BACKGROUND

The present disclosure pertains to the field of aptamer-based analyte detection assays. In particular, the present disclosure pertains to an aptamer-based electrochemical drug detection assay on a microfluidic device.

With the legalization of cannabis in certain jurisdictions, the need for cost-effective and accurate methods for detection of cannabis impairment is growing. Various assays for the specific detection of analytes, including but not limited to Tetrahydrocannabinol (THC), are known in the art. These assays include immunoassays [1], spectrophotometry [2], colorimetry [3], and mass spectrometry with chromatography [4]. However, they lack sensitivity, are affected by temperature, and require specialized training to operate these systems [5].

Biosensors have also been developed to detect analytes. Stevenson et al [6] discloses an electrochemical biosensor for detecting THC in saliva. This biosensor uses antibodies as the biosensing element. In addition to antibodies, aptamers may be used in the specific detection of analytes. Aptamers offer a number of advantages over antibodies, including but not limited to increased stability (i.e., longer shelf-life), and easier and less expensive to produce than antibodies since they do not need animals or an immune response for their production.

Aptamer electrochemical biosensors which use aptamers labelled with redox indicator molecules such as methylene blue (MB) and immobilized on an electrode are known in the art [7-10]. Upon interaction with their targets, the electrode-bound aptamers undergo conformational changes that affect electron transfer (ET) efficiency between the redox molecule and the electrode. In alternative methods which take advantage of the fact that methylene blue binds to DNA, the aptamer is not labelled with the methylene blue and the methylene blue competes with the target for binding with the aptamer.

Microfluidics is frequently used to perform liquid handling processes in the microscale that are originally executed in the macroscale and allows for different types of fluids to be manipulated and analyzed. Unfortunately, prior art adaptations of microfluidic technologies have focused on using pumps or valve-based mechanisms to manipulate the fluids [11-13]. Accordingly, it would be advantageous to provide a pumpless or valveless microfluidic device that provides the capability of both sample processing and aptamer-electrochemical detection without suffering from problems associated with moving parts and without requiring complex peripheral instrumentation to operate the device.

This background information is provided for the purpose of making known information believed by the applicant to be of possible relevance to the present disclosure. No admission is necessarily intended, nor should be construed, that any of the preceding information constitutes prior art against the present disclosure.

### SUMMARY

Aptamers configured to specifically bind to tetrahydrocannabinol and cannabidiol are disclosed, and biosensing methods and biosensor devices are described in which such aptamers are employed for detection of tetrahydrocannabinol and/or cannabidiol, with a limit of detection in the nanomolar range. In some example implementations, aptamer-based electrochemical biosensors are disclosed for sensitive and rapid detection of tetrahydrocannabinol and/or cannabidiol. Examples of microfluidic biosensors are disclosed that may be utilized in point-of-care settings.

Accordingly, in one aspect of the present disclosure, there is provided an aptamer-based sensor comprising an aptamer, the aptamer comprising a nucleic acid sequence sharing at least 50% identity with any one of SEQ ID Nos. 1-24.

In some example implementations of the aptamer-based sensor, the nucleic acid sequence shares at least 60% identity with any one of SEQ ID Nos. 1-24.

In some example implementations of the aptamer-based sensor, the nucleic acid sequence shares at least 70% identity with any one of SEQ ID Nos. 1-24.

In some example implementations of the aptamer-based sensor, the nucleic acid sequence shares at least 80% identity with any one of SEQ ID Nos. 1-24.

In some example implementations of the aptamer-based sensor, the nucleic acid sequence shares at least 90% identity with any one of SEQ ID Nos. 1-24.

In some example implementations of the aptamer-based sensor, the nucleic acid sequence shares at least 95% identity with any one of SEQ ID Nos. 1-24.

In some example implementations of the aptamer-based sensor, the nucleic acid sequence is selected from SEQ ID Nos. 1-24.

In some example implementations of the aptamer-based sensor, the nucleic acid sequence shares at least 50% identity with SEQ ID No. 22.

In some example implementations of the aptamer-based sensor, the nucleic acid sequence shares at least 60% identity with SEQ ID No. 22.

In some example implementations of the aptamer-based sensor, the nucleic acid sequence shares at least 70% identity with SEQ ID No. 22.

In some example implementations of the aptamer-based sensor, the nucleic acid sequence shares at least 80% identity with SEQ ID No. 22.

In some example implementations of the aptamer-based sensor, the nucleic acid sequence shares at least 90% identity with SEQ ID No. 22.

In some example implementations of the aptamer-based sensor, the nucleic acid sequence shares at least 95% identity with SEQ ID No. 22.

In some example implementations of the aptamer-based sensor, the nucleic acid sequence comprises SEQ ID No. 22.

In some example implementations of the aptamer-based sensor, the nucleic acid sequence shares at least 50% identity with SEQ ID No. 23.

In some example implementations of the aptamer-based sensor, the nucleic acid sequence shares at least 95% identity with SEQ ID No. 23.

In some example implementations of the aptamer-based sensor, the aptamer is bound to a working electrode and the aptamer-based sensor is an electrochemical sensor.

In another aspect, there is provided an aptamer comprising a nucleic acid sequence sharing at least 50% identity with any one of SEQ ID Nos. 1-24.

In some example implementations of the aptamer, the nucleic acid sequence shares at least 50% identity with SEQ ID No. 22.

In some example implementations of the aptamer, the nucleic acid sequence shares at least 95% identity with SEQ ID No. 22.

In some example implementations of the aptamer, the nucleic acid sequence shares at least 50% identity with SEQ ID No. 23.

In some example implementations of the aptamer, the nucleic acid sequence shares at least 95% identity with SEQ ID No. 23.

In another aspect, there is provided an artificial ligand configured to specifically bind to tetrahydrocannabinol or cannabidiol, the artificial ligand comprising a non-naturally occurring nucleic acid sequence sharing at least 50% identity with any one of SEQ ID Nos. 1 to 24.

In some example implementations of the artificial ligand, the non-naturally occurring nucleic acid sequence shares at least 50% identity with SEQ ID No. 22.

In some example implementations of the artificial ligand, the non-naturally occurring nucleic acid sequence shares at least 95% identity with SEQ ID No. 22.

In some example implementations of the artificial ligand, the non-naturally occurring nucleic acid sequence shares at least 50% identity with SEQ ID No. 23.

In some example implementations of the artificial ligand, the non-naturally occurring nucleic acid sequence shares at least 95% identity with SEQ ID No. 23.

In another aspect, there is provided a method of detecting tetrahydrocannabinol or cannabidiol in a sample, the method comprising:
contacting an electrode-bound aptamer that specifically binds tetrahydrocannabinol with a sample and a redox reagent which binds to DNA; and
determining presence of tetrahydrocannabinol or cannabidiol in the sample by measuring a current generated at the electrode, the current being associated with competitive binding of the tetrahydrocannabinol or cannabidiol and the redox reagent to the electrode-bound aptamer.

In some example implementations of the method, the electrode-bound aptamer comprises a nucleic acid sequence sharing at least 50% identity with any one of SEQ ID Nos. 1-24.

In some example implementations of the method, the electrode-bound aptamer comprises a nucleic acid sequence sharing at least 50% identity with SEQ ID No. 22.

In some example implementations of the method, the electrode-bound aptamer comprises a nucleic acid sequence sharing at least 95% identity with SEQ ID No. 22.

In some example implementations of the method, the electrode-bound aptamer comprises a nucleic acid sequence sharing at least 50% identity with SEQ ID No. 23.

In some example implementations of the method, the electrode-bound aptamer comprises a nucleic acid sequence sharing at least 95% identity with SEQ ID No. 23.

In some example implementations of the method, the redox reagent comprises methylene blue.

In some example implementations, the method further comprises determining the amount of tetrahydrocannabinol or cannabidiol in the sample by comparing current generated to a predetermined standard tetrahydrocannabinol or cannabidiol concentration curve.

In another aspect, there is provided a biosensor comprising a substrate comprising one or more microfluidic channels and an inlet in fluid communication with the one or more microfluidic channels, the one or more microfluidic channels having an assay region, the assay region comprising one or more aptamers that specifically binds to tetrahydrocannabinol or cannabidiol immobilized on an electrode.

In another aspect, there is provided a method of quantitating tetrahydrocannabinol in a sample, the method comprising:
introducing a sample suspected of containing tetrahydrocannabinol and a redox reagent which binds DNA into the inlet of the biosensor of claim 36;
allowing the sample mixed with redox reagent to flow into the assay region;
allowing any tetrahydrocannabinol or cannabidiol in the sample to bind to the aptamer; and
measuring current generated.

In another aspect, there is provided a microfluidic device, comprising:
a first layer comprising a counter electrode, a working electrode, and a reference electrode, wherein a surface of said working electrode comprises an aptamer that specifically binds tetrahydrocannabinol or cannabidiol;
a second layer comprising a fluidic channel, said second layer being interfaced with said first layer such that said fluidic channel is in fluid communication with said counter electrode, said working electrode, and said reference electrode, said second layer comprising a fluidic port in fluidic communication with said fluidic channel;
a third layer comprising a chamber for storing a buffer, said third layer further comprising an external port and an internal port in fluid communication with said chamber;
an external seal sealing said external port from an external environment, said external seal being capable of perforation by a pipette tip; and
an internal seal residing between said internal port of said third layer and said fluidic port of said second layer, such that said chamber is brought into fluid communication with said fluidic channel when said internal seal is ruptured;
wherein said third layer is configured such that when the pipette tip perforates said external seal under applied force, a distal portion of said pipette tip is brought into fluid communication with said chamber; and
wherein said internal seal is configured to rupture under applied fluidic pressure, such that injection of a sample from the pipette tip into said chamber results in rupture of said internal seal and flow of the sample and the buffer into said fluidic channel and into fluidic contact with said working electrode, said reference electrode and said counter electrode.

In some example implementations of the device, said external seal is configured such that after perforation of said external seal by the pipette tip, contact between an outer surface of the pipette tip and said external port forms an additional seal under application of an applied force.

In some example implementations of the device, said first layer, said second layer and said third layer are secured via an applied clamping force.

In another aspect, there is provided a system for providing electrochemical analysis, said system comprising:
a microfluidic device according to any one of claims 38 to 40;
a collection vessel with an opening for collection of a biological fluid sample, said collection vessel further comprising a pipette tip, said pipette tip being capable of perforation of said external seal, and wherein squeezing of said collection vessel causes evacuation of the biological fluid sample into said microfluidic device after perforation of said external seal; and
a potentiostat device connectable to said counter electrode, said working electrode and said reference electrode.

An object of the present disclosure is to provide an aptamer-based electrochemical drug detection assay. In accordance with an aspect of the present disclosure, there is provided an aptamer that specifically binds to THC and comprises the sequence of any one of SEQ ID NOs: 1 to 24.

In accordance with another aspect of the disclosure, there is provided a method of detecting THC in a sample comprising contacting an electrode-bound aptamer that specifically binds THC, with a sample and a redox reagent which binds to DNA and determining presence of THC by measuring current generated.

In accordance with another aspect of the disclosure, there is provided a biosensor comprising a substrate comprising one or more microfluidic channels and an inlet in fluid communication with the one or more microfluidic channels, the one or more microfluidic channels having an assay region, the assay region comprising one or more aptamers that specifically binds to THC immobilized on an electrode.

In accordance with another aspect of the disclosure, there is provided a microfluidic device, comprising: a first layer comprising of three electrodes: counter electrode, working electrode, and reference electrode, said electrode can be commercially available screen-printed electrodes or fabricated electrodes use for electrochemical measurement, wherein the working electrode is surface treated to contain the analyte specific aptamers; a second layer comprising a network of channels which are connected to the first layer to deliver a biological fluid to first layer; and a third layer comprising of chambers for storing buffers with an interface that is sealed to said second layer using a gasket or a flexible material, comprising a pressure seal at the said interface which perforates with application of a pipette tip and a sufficiently small opening connecting to said second layer to promote injection of the biological fluid sample.

In accordance with another aspect of the disclosure, there is provided a system for providing electrochemical analysis, said system comprising: the microfluidic device detailed above; a collection vessel with opening to collect biological sample and a pipette tip fitted with a filter, wherein, when in use, said pipette tip perforates said pressure seal and forms an air-tight seal, and said container evacuates biological fluid sample into the device by squeezing the collection vessel, and a potentiostat device to connect to said electrodes to apply currents to obtain voltammetry curves.

A further understanding of the functional and advantageous aspects of the disclosure can be realized by reference to the following detailed description and drawings.

### BRIEF DESCRIPTION OF THE DRAWINGS

Embodiments are described with reference to the accompanying drawings. In the drawings, like reference numbers can indicate identical or functionally similar elements. The drawing in which an element first appears is generally indicated by the left-most digit in the corresponding reference number.

FIG. 1 illustrates schematic representation of the THC specific Strem-18 binding aptamer used in the assay, where an 80-mer long sequence was immobilized on the gold electrodes through an alkanethiol linker. The assay exploits the change in aptamer mediated ET from the electrode to redox indicator (MB) present in solution when THC is present.

FIGS. 2A and 3B illustrate (FIG. 2A) Representative DPV data obtained with the conventional gold macro-electrode in 20 mM PBS/150 mM NaCl, pH 7.0, containing 1 μM MB with the Strem-18 aptamer before (black line) and (colored lines) after interaction with the THC at a series of concentrations: 0.1, 0.25, 0.5, 1 and 5 μM. (FIG. 2B) Dependence of the peak current (Iₚ) signal changes normalized for the surface density Γ (pmol cm⁻²) obtained in response to THC binding. Γ_{aptamer-MB}=1.8±0.3 pmol cm⁻²

FIGS. 3A, 3B and 3C illustrate (FIG. 3A) Representative CVs recorded with the conventional Strem-18 modified gold macro-electrode in 20 mM PBS/150 mM NaCl, pH 7.0, containing 1 μM MB, at the following scan rates: 0.05, 0.1, 0.25, 0.50, 0.75 and 1 V s⁻¹. The anodic and cathodic peak currents plotted versus (FIG. 3B) the scan rate and (FIG. 3C) the square root of the potential scan rate, respectively.

FIGS. 4A and 4B illustrate (FIG. 4A) Representative DPV data obtained with the conventional gold macro-electrode in 20 mM PBS/150 mM NaCl, pH 7.0, containing 1 μM MB with the Strem-18 aptamer before (black line) and (colored lines) after interaction with the THC at a series of concentrations: 1, 5, 10, 25, 50 and 100 nM. (FIG. 4B) Dependence of the Iₚ signal changes normalized for the surface density Γ (pmol cm⁻²) obtained in response to THC binding. Γ_{aptamer-MB}=5.3±0.9 pmol cm⁻²

FIGS. 5A and 5B illustrates (FIG. 5A) Representative DPV data obtained with the conventional gold macro-electrode in 20 mM PBS/150 mM NaCl, pH 7.0, containing 1 μM MB with the Strem-18 aptamer; (FIG. 5B) Dependence of the Iₚ signal changes normalized for the surface density Γ (pmol cm⁻²) obtained in response to THC binding. Γ_{aptamer-MB}=8.3±0.1 pmol cm⁻².

FIGS. 6A, 6B and 6C illustrate (FIG. 6A) Representative CVs recorded with the conventional Strem-18 modified gold macro-electrode in 20 mM PBS/150 mM NaCl, pH 7.0, containing 1 μM MB, at the following scan rates: 0.05, 0.1, 0.25, 0.50, 0.75 and 1 V s⁻¹. The anodic and cathodic peak currents plotted versus (FIG. 6B) the scan rate and (FIG. 6C) the square root of the potential scan rate, respectively.

FIGS. 7A, 7B, 7C and 7D illustrate microfluidic electrochemical system for detection of THC in patient saliva. The system uses sealed disposable fluidic components containing appropriate electrochemical buffer solutions and is designed to be compatible with low-cost, commercially available screen-printed electrodes. FIG. 7A) Image of the fabricated device using 3D-printed polylactic acid (PLA) and polydimethylsiloxane (PDMS) and secured with hex bolts. The system also uses a saliva collection vessel tube which fits a filter pipette tip used to puncture PDMS layers. The vessel is then used to apply pressure to mix samples with impregnated electrochemical buffer solution. FIG. 7B) Side-view schematic showing the impregnated and channel layer of PDMS. FIG. 7C) Assembled view of the device. FIG. 7D) Exploded view of the device.

FIGS. 8A, 8B, 8C, 8D, 8E and 8F show (FIGS. 8A, 8B and 8C) a monolithic microfluidic device with integrated electrodes and microfluidic network directly bonded on the electrodes by photolithography techniques; (FIG. 8D and FIG. 8E) Images showing working, counter and reference electrodes integrated with the microfluidic flow cell, (FIGS. 8F and 8G) Images of microfluidic channels of the prototype microfabricated by photolithography.

FIG. 9 provides a block diagram of an 8-step protocol of an embodiment of the disclosure to detect THC starting with an input (patient sample) to output (differential pulse voltammetry curve (DPV)) to measure THC concentration.

FIGS. 10A and 10B illustrate (FIG. 10A) Representative DPVs recorded in 20 mM PBS/150 mM NaCl, pH 7.0 solution containing 1 μM MB with the aptamer/MC₆OH-modified electrodes (black line) before and (colored line) after the additions of 1, 5, 10, 25, 50, and 100 nM of Δ⁹-THC. (FIG. 10B) Dependence of the Iₚ signal changes normalized for the surface density Γ (pmol cm⁻²) obtained in response to THC binding. Γ_{aptamer-MB}=4.1±0.8 pmol cm⁻².

FIGS. 11A and 11B illustrate (FIG. 11A) Representative DPVs recorded in 10% unfiltered saliva and 20 mM PBS/150 mM NaCl, pH 7.0 solution containing 1 μM MB with the aptamer/MC₆OH-modified electrodes (black line) before and (colored line) after the additions of 10, 25, and 50 nM of Δ⁹-THC. (FIG. 11B) Dependence of the Iₚ signal changes normalized for the surface density Γ (pmol cm⁻²) obtained in response to THC binding. Γ_{aptamer-MB}=3.8±0.4 pmol cm⁻².

FIG. 12 illustrates representative DPVs recorded in filtered saliva:PBS/1 μM MB (1:1 ratio) with the aptamer/MC₆OH-modified electrodes in the absence (solid line) and in the presence of 5 nM Δ⁹-THC (dashed line). Inset: the normalized Iₚ signal intensities measure in saliva:PBS/1 μM MB in the absence and in the presence of 5 nM Δ⁹-THC. Γ_{aptamer-MB}=3.8±0.4 pmol cm⁻².

FIG. 13 shows the binding affinity using fluorescence quenching of the top twenty aptamer sequences to Δ⁹-THC compared to CBD (cannabidiol) after the selection round 7, 8, and 9. Strem-18 shows the highest affinity to Δ⁹-THC and Strem-14 shows the lowest affinity to Δ⁹-THC after nine selection rounds.

FIGS. 14A and 14B show (FIG. 14A) Representative DPVs recorded with the aptamer/MC₆OH-modified electrodes in 20 mM PBS/150 mM NaCl/1 μM MB, pH 7.0 solution (black line) before and (incrementing lines) after the additions of 1, 5, 10, 25, 50, 100, 500, and 1,000 nM of Δ⁹-THC. (FIG. 14B) DPV showing the changes in measured current (normalized to background levels) in the absence and the presence of various concentrations of Δ⁹-THC (1-100 nM), where data fitting was performed using (1) the Langmuir adsorption isotherm and (2) the Scatchard model. Inset: the data normalized as logarithmic concentration. Surface density: Γ_{aptamer-MB}=1.38±0.09 pmol cm⁻².

FIG. 15 illustrates an example system for performing THC and/or CBD detection with an aptamer-based electrochemical biosensor.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

The present disclosure provides an aptamer-based tetrahydrocannabinol (THC) detection method, assay and biosensor. The THC detection method, assay and biosensor may be used to detect THC in a variety of samples including but not limited to saliva, blood and urine. THC is the primary psychoactive compound in cannabis. Accordingly, the present disclosure provides methods, assays and biosensors for use in the determination of cannabis impairment. The biosensor may be utilized in a portable device comprising the necessary components for signal processing and visualization of the sensing results. Accordingly, in certain embodiments, there is provided a device for reading the biosensors. In certain embodiments, there is provided a biosensor with a point-of-care microfluidic device.

In certain embodiments, there is provided a platform and method for the roadside determination of cannabis impairment, the platform comprises, and the method utilizes the biosensor and device for reading the biosensor described herein.

### Aptamers

The present disclosure provides aptamers that specifically bind to tetrahydrocannabinol (THC), and in some cases, other cannabinoids, such as, but not limited to, cannabidiol. As used herein, an “aptamer” refers to artificial ligand comprising a non-naturally occurring nucleic acid sequence that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An aptamer can include any suitable number of nucleotides, including any number of chemically modified nucleotides.

The aptamer may be in their natural format or may be folded to form various secondary structures, such as hairpins. The aptamers are able to specifically recognize and bind to their target molecules by drastically changing their shape acting as molecular switches to turn a sensor on and off. For small molecules, a variety of interactions can be exploited such as electrostatic interactions, or a competitive binding between the target molecule and a soluble redox indicator where the target molecule displaces the redox indicator.

Aptamers can be DNA or RNA or chemically modified nucleic acids and can be single stranded, double stranded, or contain double stranded regions, and can include higher ordered structures. An aptamer can also be a photo aptamer, where a photoreactive or chemically reactive functional group is included in the aptamer to allow it to be covalently linked to its corresponding target. An aptamer may include a tag. An aptamer can be identified using any known method, including the SELEX process. Once identified, an aptamer can be prepared or synthesized in accordance with any known method, including chemical synthetic methods and enzymatic synthetic methods.

In certain embodiments, the aptamer that specifically binds to THC comprises any of the sequences set forth below (SEQ ID NOs:1 to 24):

Strem-1 (SEQ ID NO: 1):
TGT CAC ATC TAC ACT GCT CGA AGG CGG GTT TTT
TCA CTT TTA ATT TTC ATT CAT TTT ACC TCT ATT
CAG ACA GCG TTC CC
Strem-2 (SEQ ID NO: 2):
TGT CAC ATC TAC ACT GCT CGA AGA CAG GCT GCT
TTT ATT CAT TCA TAC TTT CCT CGA TTT ACC ATT
CAG ACA GCG TTC CC
Strem-7 (SEQ ID NO: 3):
TGT CAC ATC TAC ACT GCT CGA AGC GAA TCT ACA
AGG GCT TTC TTC ATT CTC GTT CTT CCC CTT ATT
CAG ACA GCG TTC CC
Strem-8 (SEQ ID NO: 4):
TGT CAC ATC TAC ACT GCT CGA AGT CTT CCT TTT
TTA TCA TTT TTA CTT ACT CAT GTA TTT TTC ATT
CAG ACA GCG TTC CC
Strem-18 (SEQ ID NO: 5):
TGT CAC ATC TAC ACT GCT CGA AGG TCT TTC GTA
TTT GCA TTC CTC TCT TCT TCA TTT CGA GCA ATT
CAG ACA GCG TTC CC
Strem-1.1 (SEQ ID NO: 6):
CAA GTT TTT TCA CTT TTA ATT TTC ATT CAT TTT
ACC T
Strem-1.2 (SEQ ID NO: 7):
CAA TTT TTT TTT TTT AAC
Strem-18.1 (SEQ ID NO: 8):
TCT TTC GTA TTT GCA TTC CTC TCT TCT TCA TT
Strem-1.1as1 (SEQ ID NO: 9):
AAA CAA ACA AAC ATG AAT GAA AAT TAA AA
Strem-1.1as2 (SEQ ID NO: 10):
AAA CAA ACA AAC ATG AAT GAA AAA ATT AAA AAA
GT
Strem-18.1as1 (SEQ ID NO: 11):
AAA CAA ACA AAC AGG AAT GCA AAT AC
Strem-18.1as2 (SEQ ID NO: 12):
AAA CAA ACA AAC GAA TGC AAA AAT AC
Strem1.1as3 (SEQ ID NO: 13):
TTT GTT TGT TTG TTT GAA AAA ATT AAA AAA AGT
AAG TAC AAA C
Strem1.1as4 (SEQ ID NO: 14):
TTT GTT TGT TTG TTT AAA CTG AAA AAA ATT AAA
AAA GTA AGT ACA AAC
Strem 18.2as1 (SEQ ID NO: 15):
TTT GTT TGT TTG TTT CGA GCA GCA T
Strem18.2as2 (SEQ ID NO: 16):
TTT GTT TGT TTG TTT TCG AGC AGC AT
Strem-18.2 (SEQ ID NO: 17):
ATG CTG CTC GAA AAA TTC GAG CAA T
Strem-1a (SEQ ID NO: 18):
CGG GTT TTT TCA CTT TTA ATT TTC ATT CAT TTT
ACC TCT
Strem-2c (SEQ ID NO: 19):
A CAG GCT GCT TTT ATT CAT TCA TAC TTT CCT
CGA TTT ACC
Strem-7c (SEQ ID NO: 20):
C GAA TCT ACA AGG GCT TTC TTC ATT CTC GTT
CTT CCC CTT
Strem-8c (SEQ ID NO: 21):
T CTT CCT TTT TTA TCA TTT TTA CTT ACT CAT
GTA TTT TTC
Strem-18.1c (SEQ ID NO: 22):
TCT TTC GTA TTT GCA TTC CTC TCT TCT TCA
Strem-18.1c2 (SEQ ID NO: 23):
GTC TTT CGT ATT TGC ATT CCT CTC TTC TTC ATT
TCG AGC A
Strem-14 (SEQ ID NO: 24):
TAG ACA GTT CAT TTA ACT ATT CTT TTC ACT TTT
TCT CGT T.

In some example implementations, an aptamer that specifically binds to THC comprises any of the sequences set forth above (SEQ ID NOs:1 to 24).

In some example implementations, an aptamer that specifically binds to THC comprises a nucleic acid sequence selected from SEQ ID NOs:1 to 24, and/or a sequence sharing at least 50%, 55%, 60%, 65%, 70%, 75%, 80%, 85%, 90%, 95% or 99% identity with any one of SEQ ID NOs:1 to 24.

In some example implementations, an aptamer that specifically binds to THC comprises two or more copies of a nucleic acid sequence selected from SEQ ID NOs:1 to 24, and/or sequences sharing at least 50%, 55%, 60%, 65%, 70%, 75%, 80%, 85%, 90%, 95% or 99% identity with any one of SEQ ID NOs:1 to 24.

In certain embodiments, the aptamer according to the present invention may comprise at least 40 nucleotides. For example, the aptamer may comprise 40 to 80 nucleotides.

The aptamers with SEQ IDs NO: 6-24 represent the core sequences. The aptamers with SEQ IDs NO: 1 to 5 include additional nucleotides. The purpose of the additional nucleotides was to add stability by increasing the melting temperature (Tm) of the aptamer for a specific condition, in order to be successfully applied in a hybridization based aptasensor.

In some example implementations, an aptamer that specifically binds to THC may have a minimum length of 40 nucleotides, where the SEQ NOs 6-24 constitutes the core sequences of SEQ NOs 1-5.

### Nucleotides.

In some example implementations, an aptamer that specifically binds to THC may have a maximum length of 80 nucleotides. In some example implementations, an aptamer that specifically binds to THC may have a length of 40 to 80 nucleotides. In some example implementations, an aptamer that specifically binds to THC may have a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

### Methods and Assays:

The present disclosure also provides methods and assays for the detection of THC in a sample. In certain embodiments, the sample is a biological sample. In specific embodiments, the sample is selected from the group consisting of saliva, blood and urine. The method may include contacting the sample with one or more aptamers that specifically binds THC. In certain embodiments, each aptamer may comprise a nucleic acid sequence selected from SEQ ID NOs:1 to 24 and/or a sequence sharing at least 50%, 55%, 60%, 65%, 70%, 75%, 80%, 85%, 90%, 95% or 99% identity with any one of SEQ ID NOs:1 to 24.

The present disclosure also provides methods and assays for the detection of other cannabinoids, not limited to CBD in a sample. In specific embodiments, the sample is selected from the group consisting of saliva, blood and urine. The method may include contacting the sample with one or more aptamers that specifically binds to other non-THC cannabinoids with different binding efficiencies and specificity. Cross-reactivity experiments can be performed, as is known to those in the art. In certain embodiments, each aptamer may comprise a nucleic acid sequence selected from SEQ ID NOs:1 to 24 and/or a sequence sharing at least 50%, 55%, 60%, 65%, 70%, 75%, 80%, 85%, 90%, 95% or 99% identity with any one of SEQ ID NOs:1 to 24

Various methods of detecting aptamer analyte binding are known in the art and include, but are not limited to, colorimetric, fluorometric and electrochemical detection methods. Accordingly, in certain embodiments, the present disclosure provides an aptamer-based colorimetric or fluorometric THC detection method/assay. In certain embodiments, the present disclosure provides an aptamer-based electrochemical THC detection assay. In certain embodiments, the methods are conducted in real-time.

In some example embodiments, the example methods according to the present disclosure may be adapted to achieve sensitivity for target detection at low micromolar or nanomolar concentration, for example, as low as about 5 nM, or about 1 nM.

In some example embodiments, the example methods according to the present disclosure may be adapted to provide rapid detection in about 5 minutes to about 120 minutes, about 6 minutes to about 110 minutes, about 7 minutes to about 100 minutes, about 8 minutes to about 90 minutes, about 9 minutes to about 80 minutes, about 10 minutes to about 70 minutes about 15 minutes to about 60 minutes, about 20 minutes to about 50 minutes, about 25 minutes to about 40 minutes, about 5 minutes to about 40 minutes, about 5 minutes to about 30 minutes, about 5 minutes to about 25 minutes, about 5 minutes to about 20 minutes, or about 5 minutes to about 15 minutes.

In certain embodiments, there is provided a method of detecting THC in a sample, the method comprising contacting an electrode-bound aptamer that specifically binds THC, with a sample, and detecting THC in the sample by measuring current generated or a change in current generated.

In some example embodiments, the aptamer is labelled with a redox molecule such as, but not limited to, methylene blue (MB).

In some example embodiments, upon binding with THC, the electrode-bound MB labeled aptamer undergoes a conformational change and folds into a closed loop shape that affects electron transfer efficiency between the redox molecule and the electrode. Accordingly, in certain embodiments, there is provided a method of detecting THC in a sample, the method comprises contacting an electrode-bound redox molecule labeled aptamer that specifically binds THC, with a sample by increasing the observed electrochemical reduction peak of the redox label, molecule such as, but not limited to, methylene blue (MB).

In other example embodiments, the redox reagent, such as MB, is intercalated into the structure of the aptamer. Binding of THC to the structure of the aptamer, releases the intercalated redox reagent resulting in the amperometric response being decreased. Accordingly, in certain embodiments, there is provided a method of detecting THC in a sample, the method comprises contacting an electrode-bound aptamer that specifically binds THC, with a sample by competitive binding which displaces the electrostatically bound redox indicator such as, but not limited to, methylene blue (MB) forming an electric wire and decrease the observed electrochemical reduction peak of the redox indicator (MB).

In other example embodiments, some redox molecules (e.g. such as methylene blue) bind to DNA and therefore may be used in competitive binding assays. Accordingly, in certain embodiments, there is provided a method of detecting THC in a sample, the method comprises contacting an electrode-bound aptamer that specifically binds THC, with a sample and a redox reagent which binds to DNA, such as MB, and determining presence of THC by measuring current generated.

Although many of the example embodiments of electrochemical THC detection devices and methods described herein employ MB as a redox indicator, it will be understood that a wide variety of redox indicators may be employed in the alternative. In some example competitive assay applications involving competition the between the THC analyte and a redox indicator (a signal “off” aptamer-based electrochemical assay for the detection of Δ⁹-THC), the use of methylene blue may be beneficial due to its ability to bind to nucleic acids. However, it will be understood that other redox indicators may be used as well or in the alternative, such as, but not limited to: ferrocene (Fc), horseradish peroxidase (HRP), ferrocyanide (FeCN), ruthenium hexamine (RuHex), nile blue (NB), Prussian blue (PB), thionine (Th), and other azo dyes. It is expected that the analytical signal response of the aptasensor may change based on the redox indicator used. The selection of a given redox indicator may be chosen, for example, via experimentations, based on performance criteria for a given application.

In certain embodiments, the methods are quantitative. In such quantitative methods, the output generated when contacting the sample with the aptamer may be compared to output generated with specific amounts of THC to determine the amount of THC in the sample. Output with specific amounts of THC may be provided as a predetermined standard THC concentration curve.

Based on the specificity experiments the preferred aptamers are Strem-7c (SEQ ID NO:20), Strem-8c (SEQ ID NO:21), and Strem-18c (SEQ ID NO: 22). For multiplexing purposes, the combination of two aptamers in a single assay is preferred where two or more cannabinoids are measured simultaneously, the shorter (truncated) aptamers are warranted: Strem-18 1c2 (SEQ ID NO: 23) and Strem-14 (SEQ ID NO:24).

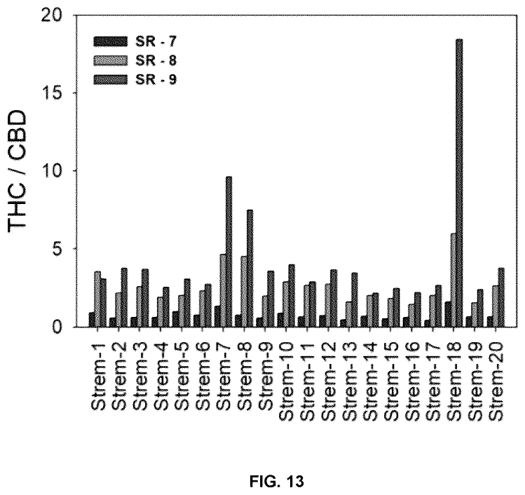

In vitro selection of aptamers against Δ⁹-THC specific was achieved with FRELEX where selection was performed for 9 selection rounds, using a library of oligonucleotides consisting of a random region of 40 nucleotides (10¹⁵ sequences). All oligos from selection round 7 to 9 were processed and the specificity of the top twenty most abundant sequences against Δ⁹-THC versus the frequency of the same sequences against CBD (cannabidiol) were further analyzed by fluorescence quenching with graphene oxide as presented in FIG. 13.

It will be understood that a wide variety of electrochemical detection modalities may be employed to detect THC using an aptamer-based electrochemical senor. Non-limiting example methods include cyclic voltammetry (CV), chronoamperometry (CA), chronopotentiometry (CP), chronocoulometry (CC), differential pulse voltammetry (DPV), square wave voltammetry (SWV), electrochemical impedance spectroscopy (EIS).

While many of the preceding example embodiments relate to the use of a THC aptamer for diagnostics assays that employ electrochemical detection, it will be understood that the present example aptamers may be employed for a wide range of uses and applications. For example, the example aptamers disclosed herein (and variations thereof) may be implemented with other systems in conjunction with other biosensing elements, such as antibodies or engineered microbes, additional assays can involve fluorescence, chemiluminescence, colorimetric detection or fluorescence resonance energy transfer (FRET). The aptamer can be easily labelled with a reporter, and can act to signal the presence or absence of the target molecule (e.g., Δ⁹-THC). Such reporter molecules may be a fluorophore-labelled aptamer, or a fluorophore-quencher labeled aptamer for a signal “on” or signal “off” approach.

### Biosensors

The present disclosure further provides example biosensors comprising one or more aptamers that specifically bind THC. The biosensor may be utilized in a portable device, optionally handheld, comprising the necessary components for signal processing and visualization of the sensing results. Accordingly, in certain embodiments, the present disclosure provides a device for reading the biosensor. In such embodiments, the biosensor may be used in the roadside determination of cannabis impairment. In certain embodiments, there is provided a biosensor with a point-of-care microfluidic device.

In certain embodiments, there is provided a biosensor comprising a substrate comprising one or more connected microfluidic channels and an inlet in fluid communication with the one or more microfluidic channels, the one or more microfluidic channels having an assay region, the assay region comprising one or more aptamers that specifically binds to THC immobilized on an electrode.

A biosensor according to the present disclosure may include at least one aptamer comprises a nucleic acid sequence selected from SEQ ID NOs:1 to 24 and/or a sequence sharing at least 50%, 55%, 60%, 65%, 70%, 75%, 80%, 85%, 90%, 95% or 99% identity with any one of SEQ ID NOs:1 to 24.

### First Example Microfluidic Device Configuration

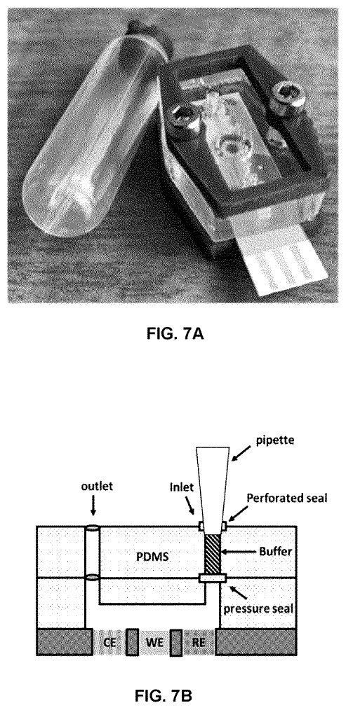

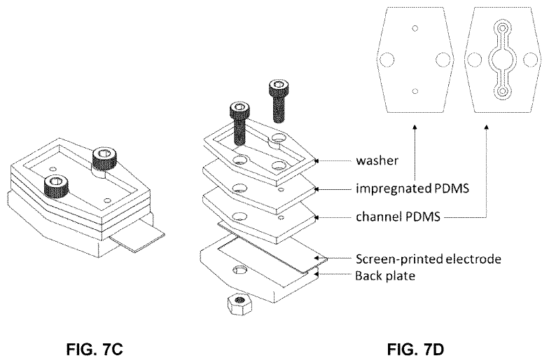

An aptamer-based THC sensor may be implemented according to a wide range of device modalities and configurations, as described in detail below. One example configuration is illustrated in FIGS. 7A-7D.

As shown in the figures, the example device includes a plurality of device layers, including a first layer comprising of three electrodes: counter electrode, working electrode, and reference electrode. The electrodes can be commercially available screen-printed electrodes or fabricated electrodes for electrochemical measurement. The working electrode is surface treated to contain aptamers that specifically bind the target analyte such as THC. In specific embodiments, the aptamers secured to the working electrode comprises at least one aptamer selected from SEQ ID NOs:1 to 24 and/or a sequence sharing at least 50%, 55%, 60%, 65%, 70%, 75%, 80%, 85%, 90%, 95% or 99% identity with any one of SEQ ID NOs:1 to 24.

The device also includes a second layer comprising at least one fluidic channel which is connected to the first layer to interface the biological fluid to the first layer.

A third layer includes a chamber for storing buffers. This chamber is enclosed by two seals: a top seal which protects the device from the outside environment and is meant to be perforated when the device is used, and a pressure seal comprised of a cleft flexible material which expands at the interface with application of pressure and releases stored and applied fluid into to the second layer. Non-limiting examples of suitable materials for forming the seals include Cyclic olefin copolymer (COC), polyamide, polyester-based, Polyvinylidene Chloride, polyethylene, polyolefin. When sample is added to the device using a pipette, the pipette tip perforates the top seal and fluid pressure forces liquid to exit through the pressure seal into the second layer. The top port of the third layer (where the outer seal resides) can take on any two dimensional geometry but not limited to circular, rectangular or square. In some example embodiments, the top port of the third layer (where the outer seal resides) can be shaped so that when the pipette tip perforates the seal and extends into the port, a seal can be formed with the outer surface of the pipette tip under an applied force, thereby enabling the application of fluidic pressure when delivering the sample into the chamber, such that the sample and the buffer in the chamber mix and are delivered into the fluidic channel within the second layer and contact the electrodes.

In certain embodiments, the device layers could be fabricated as separate pieces attached together using adhesive or clamping mechanisms such as bolts, clips, pressure-fittings or brick-and-knob connection. FIGS. 7A-7D illustrate an example implementation in which device layers are clamped together using bolts. Non-limiting example materials for forming any of the device layers include PDMS, PMMA, bisphenol A epoxy or SU8, acrylonitrile butadiene styrene, polylactic acid, polyethylene terephthalate glycol, thermoplastic polyurethane, polyvinyl alcohol, polystyrene, ceramic-based materials, thermoset polyester, polycarbonate, polyethylene glycol diacrylate, and perfluorinated compounds.

In alternative embodiments described below, a device configuration may comprise of the channel network bonded to a metal-on-glass sensing electrode device, or sensing electrodes inserted directly into the channels.

FIG. 7A shows an experimental realization of the example device that consists of a microfluidic channel layer, an assay region, and cover containing inlets and outlets made in polydimethylsiloxane (PDMS) which embeds a commercially available screen-printed electrode (SPE), and secured by 3D-printed (polylactic acid) top and backplate washers.

The gold SPE electrode was modified as mentioned above and gently placed between the backplate and the PDMS layer. The microfluidic device was assembled by layering the back plate, SPE, PDMS channels, and washer followed by fastening using two hex socket cap screws and nuts. To make a seal between the PDMS and electrodes the screws were made finger-tight with the appropriate hex key.

In other example embodiments, the electrochemical sensing elements can be integrated within a microfluidic channel network. For instance, the aptamer-modified electrodes can be produced from commercially available SPEs and can be produced by photolithography microfabrication.

### Second Example Microfluidic Device Configuration

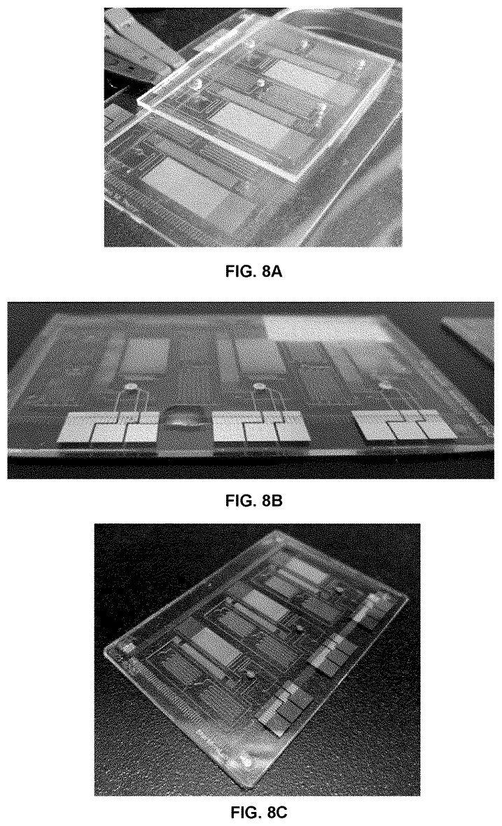

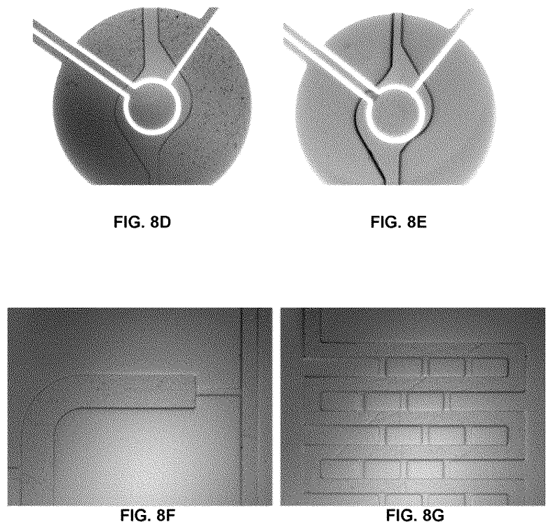

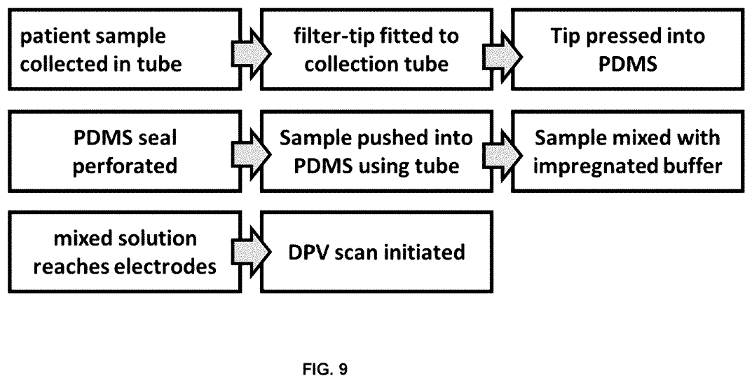

An example implementation of such a device configuration is illustrated in FIGS. 8A-8F. FIGS. 8A-8C are different views of the device. The device consists of a two layers. By etching metal-on-glass, the first layer was obtained comprising of electrochemical sensing element: counter electrode, working electrode, and reference electrode. The second layer containing the microfluidic network (comprised by microfluidic channel and assay region connected to inlet and outlet channels) can be integrated to first layer containing the aptamer-modified sensing element on a: a) monolithic multilayer approach, directly bonded on the metal-on-glass first layer by photolithography; and on a b) modular multilayer approach where electrodes can be inserted directly into the channel network. The latter with independent microfluidic layers situated on the metal-on-glass electrode layer, can be produced by i) fast prototyping (3D-printing, micromilling, replication) microfabrication and/or ii) by soft lithography.

FIG. 8D is an image showing working, counter and reference electrodes integrated with the microfluidic channel. The electrode is an exposed layer with an overlayed fluidic channel.

The designed microfluidics chamber can be used as: (i) multi-use system (reusable) or (ii) as a single-use (disposable) cartridge system. In certain embodiments in a multi-use system, the electrochemical detection of THC is determined by adding pre-mixed sample (e.g. 500-0.1 μL) containing THC, MB, saliva and buffer into the inlet port. For adequate binding of the aptamer to THC in sample, the device was incubated for 10 minutes at room temperature. Measurements are obtained by connecting the electrodes to the potentiostat system and trigging a DPV cycle using the potentiostat software. Parameters used for DPV measurements are detailed below. After the measurements, the sample was pushed out through the outlet port with a pipette.

The microfluidic chamber may also be used with a pressure seal, where the buffer and redox reagent are pre-stored in the channel layer as a single-use cartridge system which is combined in-tandem with the low-cost SPE which are modified with our targeting aptasensor. Overall, the modular system will allow the user a simple path to prepare the device for multiple use, while the use of commercial SPE designs allow for a simple integration into commercial potentiostat systems and software for an elevated user experience.

### System Configuration

In certain embodiments, there is provided a system for providing electrochemical analysis, said system comprising: the microfluidic device described above, the collection vessel described above and a potentiostat device connected to said electrodes to apply currents to obtain voltammetry curves.

In certain embodiments, there is provided a method of quantitating THC in a sample, the method comprising: introducing a sample suspected of containing THC and a redox reagent which binds DNA into the inlet of a biosensor as detailed above. Allowing the sample mixed with redox reagent to flow into the assay region, allowing any THC in the sample to bind to the aptamer and measuring current generated.

### Applications

While the present disclosure illustrates the implementation of THC aptamer-based sensing devices in some example microfluidic platforms, it will be understood that these example platforms are not intended to be limiting. Indeed, a wide variety of microfluidic and microfluidic modalities may be employed to interface samples with aptamer-based transducers, including, but not limited to, droplet or digital microfluidics, lateral flow, or bulk-flow in channels such as in a flow cell. In other example implementations, an aptamer-based THC assay can also be implemented in microwells (for example, using fluorophore-quencher labeled aptamers).

The present example embodiments may be employed for and/or adapted to a wide range of applications (use-cases), including, but not limited to, point-of-care drug testing in industrial, clinical, or roadside settings. Additionally, this aptasensor could be used for analysis in a chemical production setting such as natural (plant-based) or synthetically derived (organic synthesis, synthetic biology) cannabinoids. In the case of synthetic biology, the aptasensor could be used to screen for microbes engineered to produce cannabinoids since currently, companies producing cannabinoids rely on LC-MS for analysis.

The results described in the Examples below have focused on the non-limiting point-of-care application in which 50% filtered saliva is used for analysis. For a simple point-of-care testing platform, testing cannabinoids in saliva is the least intrusive compared to blood or urine testing, however, the present inventors have also shown elective analysis using mice serum samples as a proof of concept. Using the aptasensor platform in hospitals, other institutions or homes, other test samples sources such as blood, serum or urine can be used. For a continuous real-time testing, sweat could also be employed as a suitable sample type.

### Example System

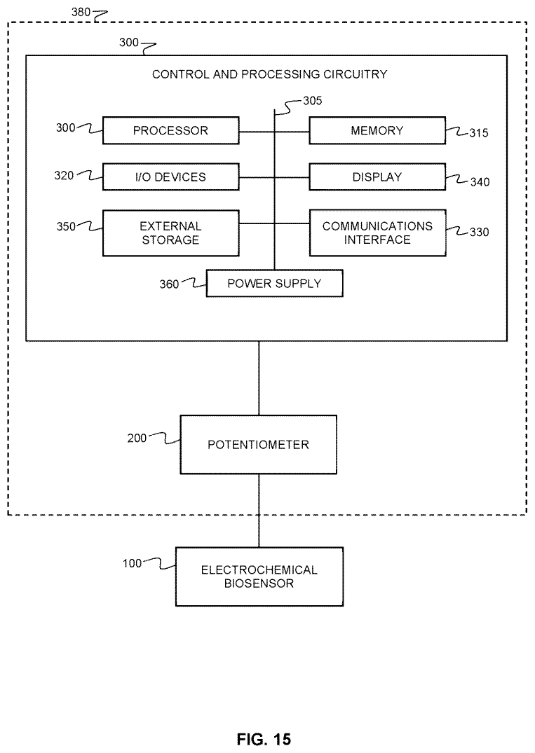

Referring now to FIG. 15, an example system is shown that includes an aptamer-based electrochemical biosensor 100 that is operatively coupled to potentiometer 200 and control and processing circuitry 300. The aptamer-based electrochemical biosensor 100 may be based, for example, any of the preceding example embodiments, or variations thereof.

As shown in the example embodiment illustrated in FIG. 15, the control and processing circuitry 300 may include a processor 310, a memory 315, a system bus 305, one or more input/output devices 320, and a plurality of optional additional devices such as communications interface 330, display 340, external storage 350, and power supply 360.

The present example methods for controlling the operation of the electrochemical sensor can be implemented via processor 310 and/or memory 315, e.g. controlling the potentiometer 200 to perform an electrochemical detection method, such as, but not limited to, cyclic voltammetry.

The methods described herein can be partially implemented via hardware logic in processor 310 and partially using the instructions stored in memory 315. Some embodiments may be implemented using processor 310 without additional instructions stored in memory 315. Some embodiments are implemented using the instructions stored in memory 315 for execution by one or more microprocessors. Thus, the disclosure is not limited to a specific configuration of hardware and/or software.

It is to be understood that the example system shown in the figure is not intended to be limited to the components that may be employed in a given implementation. For example, the system may include one or more additional processors. Furthermore, one or more components of control and processing circuitry 300 may be provided as an external component that is interfaced to a processing device. For example, one or more components of the control and processing circuitry 300 may be integrated with the potentiometer 200, as shown at 380 (or even integrated with the electrochemical biosensor 100). In another example implementation, the control and processing circuitry 300 may be wirelessly connected to the potentiometer 200, for example, through a wireless communication modality such as Wifi or Bluetooth®.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed herein can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Tetrahydrocannabinol (THC) Detection Through Specific Aptamer Binding in an Electrochemical Assay

### Introduction

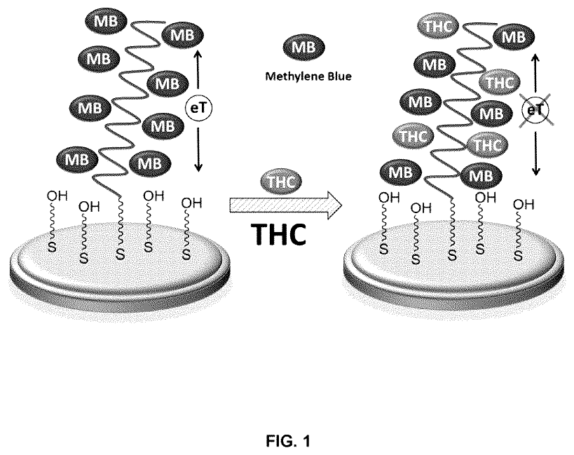

The present example demonstrates tetrahydrocannabinol (THC) detection through specific aptamer binding in an electrochemical assay. The aptamer sequence was obtained by SELEX. In this non-limiting example, an 80-mer long called Strem-18 aptamer (TGT CAC ATC TAC ACT GCT CGA AGG TCT TTC GTA TTT GCA TTC CTC TCT TCT TCA TTT CGA GCA ATT CAG ACA GCG TTC CC) was utilized in the THC binding assay. Based on the length of the aptamer, we decided to move toward a signal “on-off” type of biosensor. In this approach, an unlabeled Strem-18 aptamer was used in the presence of soluble methylene blue (MB) redox indicator. Due to the competitive binding between THC and MB, it is expected that there would be a signal decrease in the measured analytical signal based on the concentration of the THC, see FIG. 1. On the other hand, the binding would be performed in situ, which would further simplify the entire procedure compared to the direct modification on the electrode surface.

### Materials

Tetrahydrocannabinol (THC), Methylene blue (MB), 6-Mercapto-1-hexanol (MCO₆H), Tris(2-carboxyethyl) phosphine hydrochloride (TCEP), Potassium phosphate monobasic (KH₂PO₄), Potassium phosphate dibasic (K₂HPO₄), Sodium chloride (NaCl), and Polydimethylsiloxane (PDMS) were all purchased from Sigma-Aldrich. All solutions were prepared by using Milli-Q water (18 MΩ, Millipore, Bedford, MA, USA). The THC specific binding aptamer (Strem-18, 80-base sequence) with a 5′ C₆-disulfide modification (HOC₆-S-S-C₆-5′-TGT CAC ATC TAC ACT GCT CGA AGG TCT TTC GTA TTT GCA TTC CTC TCT TCT TCA TTT CGA GCA ATT CAG ACA GCG TTC CC-3′) was synthesized by Metabion International AG, Martinsried, Germany. Polylactic acid (PLA) 3D printer filament (blue −2.85 mm) was purchased from Innofil3D.

### Cleaning and Modification of Gold Disk Electrodes and Screen-Printed Electrodes (SPE)

Gold disk electrodes (CH Instruments, Austin, TX; diameter 1.6 mm) were cleaned as followed: first they were cycled in 0.5 M NaOH between 0.0 and −1.6 V (10 cycles, 0.1 V s⁻¹) and then polished on a micro-cloth pad in 1 μm and then 0.1 μM alumina slurries, respectively, followed by washing with water and ultra-sonication in 1:1 ethanol-water solution for 10 min. Next, the electrodes were washed with water and electrochemically polished stepwise in 1 M H₂SO₄ (from −0.3 to 1.7, 10 cycles, 0.3 V s⁻¹) and 1 M H₂SO₄/10 mM KCl (from 0 to 1.7, 10 cycles, 0.3 V s⁻¹). Electrochemical surface area of the electrode was estimated after this step by integration of the gold surface oxide reduction peak in 0.1 M H₂SO₄ and by using a conversion factor of 390 μC cm⁻² [14]. The electrodes were then thoroughly washed with Milli-Q water and kept in absolute ethanol before aptamer immobilization.

Prior of electrode modification, a mixture of 50 nM thiol-modified Strem-18 aptamer and TCEP in 20 mM PBS/150 mM NaCl, pH 7.0 was prepared and left for 1 h at room temperature (rt) for disulfide bond reduction. For preparation of self-assembled aptamer monolayers, a 10 μL drop of the freshly prepared mixture was placed onto the clean gold electrode surface. The electrodes were covered with an Eppendorf vial and left overnight at rt. Next day the aptamer-modified electrodes were rinsed with 20 mM PBS/150 mM NaCl, pH 7.0, and exposed to 10 mM MC₆OH solution in the same buffer solution for 30 min. The aptamer-modified electrodes were subsequently placed in 20 mM PBS/150 mM NaCl, pH 7.0, and kept in the same solution until used in the electrochemical experiments.

Screen-printed electrodes (SPE) in a three-electrode system with gold working (ϕ=2 mm), gold counter and silver pseudo reference on a flexible polyester support material were purchased from PalmSens B.V. (Cat. No. IS-W1-2.C1.RS.35) (Houten, the Netherlands). The SPE were cleaned according to Shanmugam et al [15] in a single step, where they were cycled in a drop of 0.5 M H₂SO₄ between −1.0 V and +1.3 V (12+12 cycles, 0.1 V s⁻¹). The ECSA of the planar-gold SPE were estimated by integration of the gold surface oxide reduction peak in 0.5 M H₂SO₄ obtained in a conventional three-electrode cell (Ag/AgCl (3M KCl) as reference and platinum wire as counter) by using a conversion factor of 390 μC cm⁻² [14]. The SPE were then thoroughly washed with Milli-Q water before aptamer immobilization. The aptamer immobilization followed the same protocol as described above, except the droplet covered gold SPE was left for overnight incubation at rt in a closed Petri dish containing a small volume of 20 mM PBS/150 mM NaCl, pH 7.0 inside to avoid evaporation.

### Analysis of Aptamer Surface Coverage

The surface coverage was also estimated from the MB signal associated with the duplexes formed and the MB surface coverage (Γ_{DNA-MB}) was estimated according to the following equation:

$${Γ}_{DNA - MB} = Q / ( nFA ) \qquad ( 1 )$$

where Q is the charge (C), obtained by integration of the cathodic peak area, n is the number of electrons involved in MB reduction, F is the Faraday number (C mol⁻¹) and A is the electrochemical surface area of the gold electrode (cm²).

### Instrumentation

Cyclic voltammetry (CV) and differential pulse voltammetry (DPV) were carried out in a conventional three-electrode cell with a PalmSens 4 electrochemical system (PalmSens B.V., Houten, the Netherlands) equipped with PSTrace 5 (version 5.8). Ag/AgCl (3M KCl) and platinum wire was used as the reference and counter electrodes, respectively. In DPV, the modulation amplitude was 40 mV, step potential: 10 mV and apparent scan rate: 20 mV s⁻¹. The THC binding to the aptamer was performed in situ by adding different concentrations of THC, directly in the electrochemical cell with 20 mM PBS/150 mM NaCl, pH 7.0. containing 1 μM MB. The screen-printed electrodes (SPE) were exposed to 20 μL of the THC containing samples directly on their surface followed by DPV under the previously mentioned conditions. All measurements were performed after 10 min incubation of the aptamer-modified electrodes in the THC- and MB-containing working solutions at rt inside a Faraday cage (NB: the working solutions were not de-aerated).

### Results

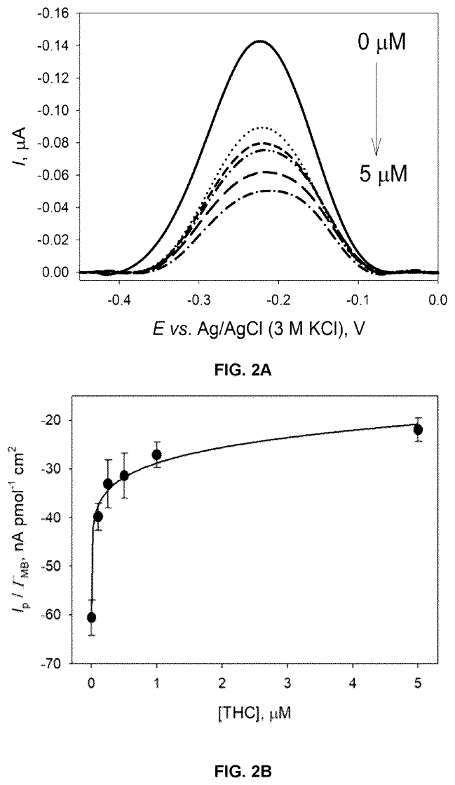

The electrodes cleaning protocol (see section above) needs to be applied to obtain reproducible electrode surfaces prior to electrochemical detection of THC. Next, we studied the performance of the proposed electrochemical, using the signal-off approach in the micromolar concentration range, and the results are presented in FIGS. 2A and 2B, where the measurements were performed in situ, with a 10-minute THC binding time. It can be observed that the Strem-18 based aptamer biosensor produced pronounced voltametric MB signals at −217±9 mV. The DPV signals showed a slight peak shift toward positive potentials due to THC binding. We constructed calibration curves based on the DPV signals, by plotting the peak currents (Iₚ) normalized for the aptamer surface coverage, see FIG. 2B. As expected, the signals decreased during THC binding due to the competitive binding with MB. The THC binding showed saturation over 1 μM, while the significant signal drop between 0 and 0.1 μM suggested that lower concentrations of THC binding to the Strem-18 aptamer is detectible in these specific conditions.

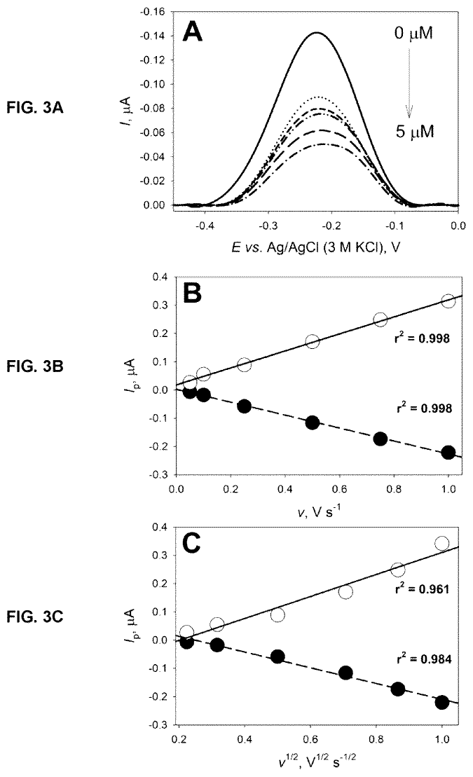

Next, the electrochemical behavior of MB on the Strem-18 aptamer modified electrodes was studied with 1 μM MB. At scan rates lower than 1 Vs⁻¹ a well-developed couple of peaks with a mid-potential at around −270 mV could be followed, see FIG. 3A. At higher scan rates (results not shown), the CVs were distorted due to the limitations of the potentiostat, therefore there were not taken into further consideration in these studies. Consistently, the MB peak currents showed linear dependence on the potential scan rate, characteristic of ET in the adsorbed state, see FIG. 3B, 3C.

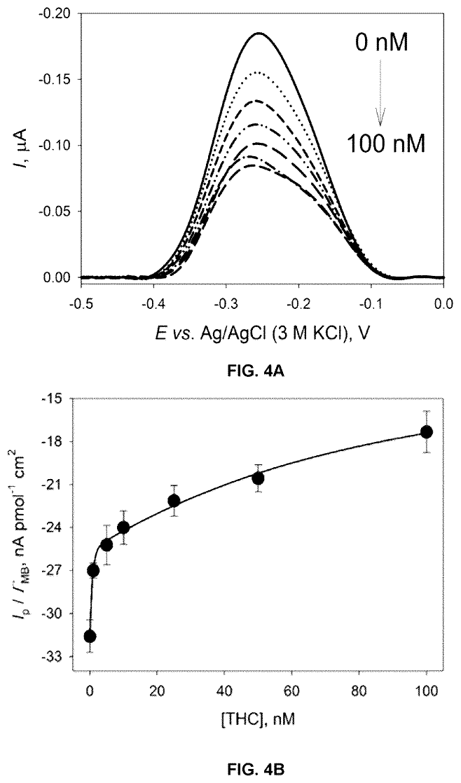

After demonstrating sensitivity in the micromolar concentration range we focused on lowering the detection limit further. We studied the THC binding toward the Strem-18 aptamer in the nanomolar concentration range, see FIGS. 4A and 4B, where the biosensor produced well-developed voltametric MB signals. The DPV signals showed a decrease in their intensity during the THC binding, see FIG. 4A, however, the peak shift towards a more positive potentials were less pronounced compared to the studies performed in the micromolar concentration range. The calibration curve obtained showed the possibility of detecting 1 nM of THC binding, see FIG. 4B.

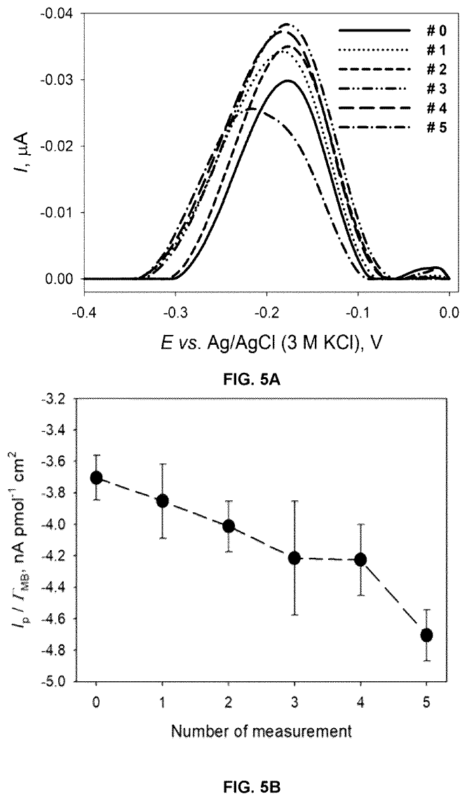

Due to the small peak shift observed during the THC binding to the Strem-18 aptamer, we decided to perform control studies where several DPVs were recorded in 1 μM MB, without the THC addition and the results are shown in FIG. 5A. It can be observed that the DPV signals showed a variation between the measurements, however, the variation was indeed small. In order to determine the actual difference between the measurements, we normalized the peak currents for the DNA surface coverage, see FIG. 5B. It can be observed that the peak current gradually decreased, however, the highest decrease was around 1.1 nA pmol⁻¹ cm² after several measurements performed, which is significantly smaller compared to the signal decrease of 5.0 nA pmol⁻¹ cm² observed after the addition of 1 nM of THC to the electrolyte.

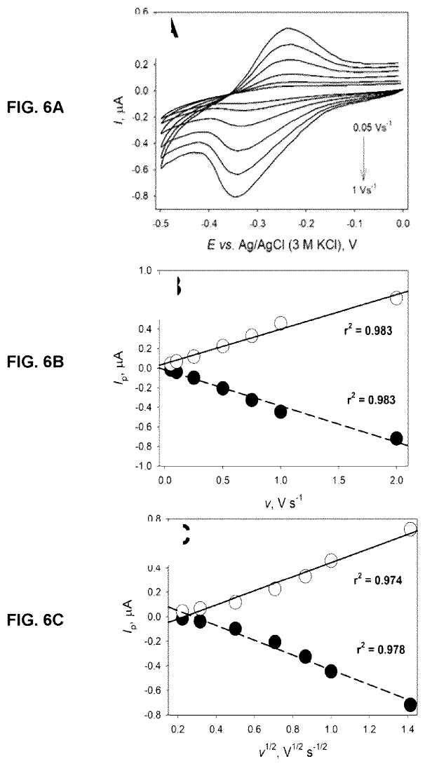

Next, the electrochemical behavior of MB on the Strem-18 aptamer modified electrodes was studied with 1 μM MB, see FIGS. 6A-6C. The MB peak currents showed linear dependence on the potential scan rate, characteristic of ET in the adsorbed state, see FIGS. 6A-6C.

For autonomous THC detection, we have developed a portable microfluidic platform which takes advantage of commercially available screen-printed electrodes (SPE). This system prototype consists of 3D-printed fastening hardware called washer and back plate, which applies pressure to form a liquid-tight seal between a PDMS channel layer and the SPE, see FIGS. 7A-7D.

The block diagram of an 8-step protocol of an embodiment of the disclosure to detect THC starting with an input (patient sample) to output (differential pulse voltammetry curve (DPV)) to measure THC concentration is presented in FIGS. 8A-8F. First the patient sample is collected into a tube which is fitted by a filter-tip for saliva pretreatment. The tip is pressed into the PDMS and perforated while the samples is pushed into the PDMS. Next, the sample is mixed with the impregnated buffer and this mixture will reach the three-electrode screen-printed system. Finally, a DPV scan is triggered by a computer to read the analytical signal.

A different prototype of a microfluidic device with integrated electrodes within the microfluidic network has been also fabricated by photolithography microfabrication techniques to prove the monolithic integration of sensing elements and fluidic assay region, see FIGS. 8A-8F. Metal-on-glass etching was applied to obtain the first layer comprising of electrochemical sensing element: counter electrode, working electrode, and reference electrode. The second layer containing the microfluidic network (comprised by microfluidic channel and assay region connected to inlet and outlet channels) was integrated to first layer containing the aptamer-modified sensing element on monolithic multilayer approach, directly bonded on the electrodes first layer by photolithography in SU-8 (FIGS. 8A-8F). A cover layer in PDMS with inlet and outlet channels can seal the microfluidic network to operate the prototype.

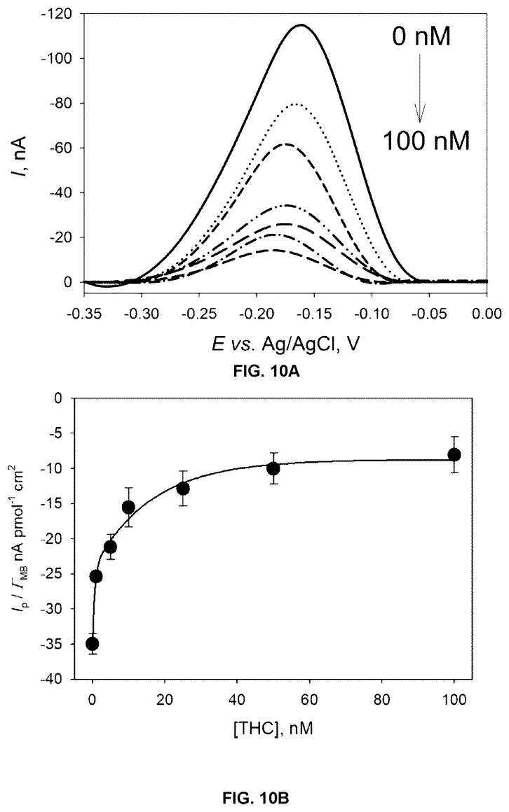

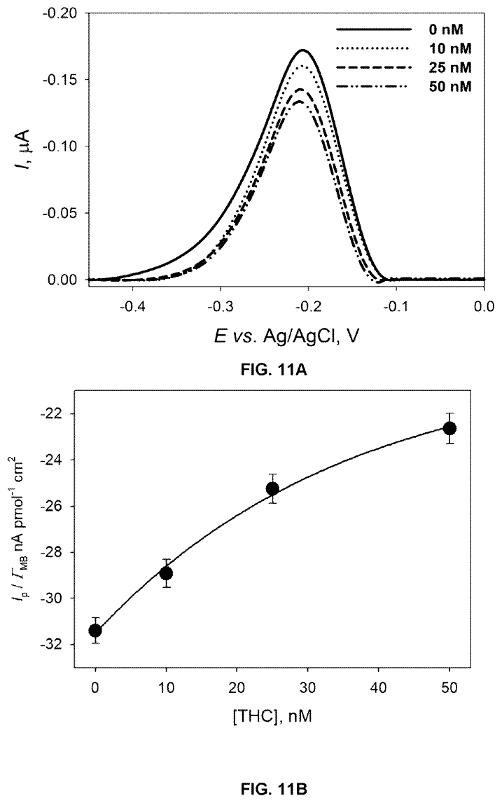

The analytical response of the aptamer/MC₆OH-modified screen-printed electrodes (SPE) electrodes in conjunction with the fabricated microfluidic chamber was studied, where representative DPV's recorded in recorded in 20 mM PBS/150 mM NaCl, pH 7.0 solution containing 1 μM MB in absence and presence of various concentrations (1-100 nM) of Δ⁹-THC are presented in FIG. 10A. The measured cathodic DPV currents showed a gradual decrease upon the addition of Δ⁹-THC in the clinically relevant concentration range. The calibration curves constructed based on the DPV responses by plotting the Iₚ signal changes normalized for the aptamer surface coverage, see FIG. 10B, showed that the signals decreased during THC binding due to the competitive binding with MB. The aptasensor equipped with the microfluidic chamber could clearly detect the presence of 1 nM of Δ⁹-THC in PBS. Next, we evaluated the real-life applicability of the aptamer/MC₆OH-modified electrochemical assay as a possible point-of-care device to detect Δ⁹-THC in biological fluids. To this end we performed electrochemical studies on modified gold SPE using reduced sample volumes in the presence of untreated human saliva with spiked Δ⁹-THC (10-50 nM) in 20 mM PBS/150 mM NaCl/1 μM MB, pH 7.0, where the representative DPVs are presented in FIG. 11A. The calibration curves constructed based on the DPV responses by plotting the Iₚ signal changes normalized for the aptamer surface coverage, see FIG. 11B, showed that the signals decreased during THC binding, however, the presence of 10% saliva has a significant influence on the detection limit of the aptasensor which showed a 10-fold increase.

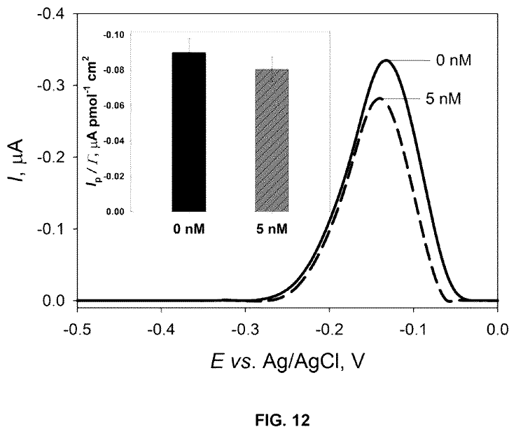

Based on the aptasensor performance obtained in the presence of untreated saliva, we investigated the use of a 10 kDa size exclusion centrifugation column in order to eliminate interfering elements present in saliva such as microorganisms, proteins, and lipids. Representative DPVs recorded in 1:1 ratio of filtered saliva alongside 20 mM PBS/150 mM NaCl/1 μM MB, pH 7.0, with the aptamer/MC₆OH-modified SPE electrodes in the absence and in the presence of 5 nM Δ⁹-THC are shown in FIG. 12. The presence of 5 nM Δ⁹-THC in the saliva clearly produced a drop in the measured cathodic DPV currents, while measurements performed with multiple aptasensor platforms showed an average of a 1.1-fold decrease in the cathodic DPV currents normalized for the surface density, see FIG. 12, inset.

### CONCLUSION

The present example demonstrate the detection of THC using an unlabeled specific aptamer sequence for THC binding in an electrochemical setup and MB as a redox indicator, exploiting the competitive binding between the two. The assay showed a robust and sensitive performance toward THC binding in the micromolar concentration range with a detection limit of 0.1 μM. The detection limit could be decreased to 1 nM, however, the assay was less robust and depended on the surface coverage. Additionally, a modular prototype of a point-of-care microfluidic device was provided that is designed to ease the use of the aptamer-based analyte detection assay. The proposed assay also demonstrated simplicity by performing the THC addition in situ instead on the electrode surface, while the binding time was also significantly reduced to 10 minutes. Measurements performed in small volume samples with the aptamer-modified screen-printed electrodes combined with the microfluidics chamber showed that the presence of unfiltered (10%) saliva had a 10-fold increase in the LOD of the aptasensor. On the other hand, pretreatment of saliva by filtration improved the LOD to 5 nM of Δ⁹-THC in a 1:1 ratio of saliva to PBS. The results clearly demonstrate the feasibility regarding the applicability of the electrochemical aptasensor towards a rapid point-of-care platform for the detection of Δ⁹-THC in saliva.

### Example 2: CBD (Cannabidiol) Detection

In vitro selection of aptamers against Δ⁹-THC specific was achieved with FRELEX, where selection was performed for 9 selection rounds, using a library of oligonucleotides consisting of a random region of 40 nucleotides (10¹⁵ sequences). The top twenty most abundant sequences were further analyzed for their specificity by comparing the enrichment in selection rounds 7, 8, and 9 against Δ⁹-THC versus CBD (cannabidiol), resulting in aptamers by using fluorescence quenching (FIG. 13). The results show that aptamers Strem-7 (SEQ ID NO:3), Strem-8 (SEQ ID NO:4), and Strem-18 (SEQ ID NO:5) exhibited the highest specificity (FIG. 13). On the other hand, Strem-14 (SEQ ID NO:2) showed the least affinity towards Δ⁹-THC and showed a higher affinity towards CBD.

### Example 3: Short Aptamer without Linkers

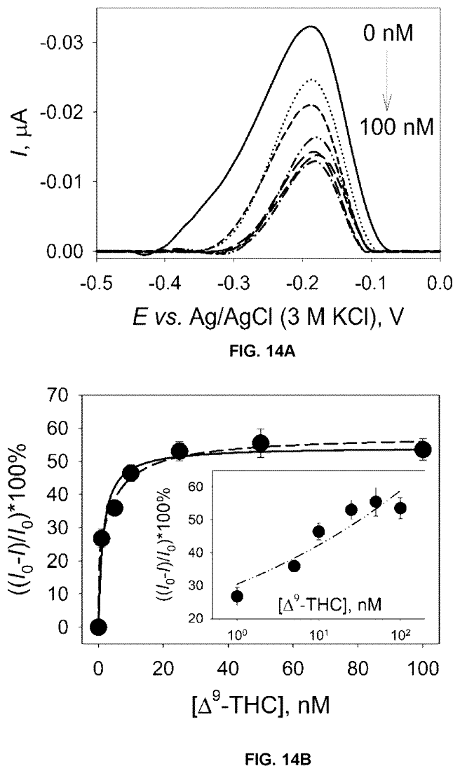

Representative DPVs obtained with the Strem-18 1c2 (SEQ ID NO:23) modified electrodes for various concentrations (0-100 nM) of Δ⁹-THC in PBS and MB, shows a gradual decrease upon the addition of THC in the clinically relevant concentration range (FIG. 14A). The calibration curves constructed based on the DPV responses and fitted by using the Langmuir adsorption isotherm and the Scatchard model showed higher value for K_{b} (4.87±0.54) and a lower value for K_{d} (1.83±0.31 nM) (FIG. 14B), with a determined LOD of 1 nM and a sensitivity of (7.00±1.44)10⁻¹⁰ A nM⁻¹. These values are in agreement with the tests performed with SEQ ID 5 showing that the shorter sequences can bind to THC.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### REFERENCES CITED

1. Huestis, M. A., J. M. Mitchell, and E. J. Cone, Detection times of marijuana metabolites in urine by immunoassay and GC-MS. J. Anal. Toxicol., 1995. 19(6): p. 443-449.
2. Hazekamp, A., et al., Chromatographic and spectroscopic data of cannabinoids from Cannabis sativa L. J. Liq. Chromatogr. Relat., 2005. 28(15): p. 2361-2382.
3. dos Santos, N. A., et al., Evaluating the selectivity of colorimetric test (Fast Blue BB salt) for the cannabinoids identification in marijuana street samples by UV-Vis, TLC, ESI (+) FT-ICR MS and ESI (+) MS/MS. Forensic Chem., 2016. 1: p. 13-21.
4. Salomone, A., et al., Simultaneous analysis of several synthetic cannabinoids, THC, CBD and CBN, in hair by ultra-high performance liquid chromatography tandem mass spectrometry. Method validation and application to real samples. J. Mass Spectrom., 2012. 47(5): p. 604-610.
5. Klimuntowski, M., et al., Electrochemical sensing of cannabinoids in biofluids: a noninvasive tool for drug detection. ACS Sens., 2020. 5(3): p. 620-636.
6. Stevenson, H., et al., A rapid response electrochemical biosensor for detecting THC in saliva. Sci. Rep., 2019. 9(1): p. 1-11.
7. Xiao, Y., et al., A reagentless signal-on architecture for electronic, aptamer-based sensors via target-induced strand displacement. J. Am. Chem. Soc., 2005. 127(51): p. 17990-17991.
8. Lai, R. Y., K. W. Plaxco, and A. J. Heeger, Aptamer-based electrochemical detection of picomolar platelet-derived growth factor directly in blood serum. Anal. Chem., 2007. 79(1): p. 229-233.
9. Ferapontova, E. E. and K. V. Gothelf, Optimization of the Electrochemical RNA-Aptamer Based Biosensor for Theophylline by Using a Methylene Blue Redox Label. Electroanalysis, 2009. 21(11): p. 1261-1266.
10. Catanante, G., et al., Sensitive analytical performance of folding based biosensor using methylene blue tagged aptamers. Talanta, 2016. 153: p. 138-144.
11. Zhang, Y., T.-M. Tseng, and U. Schlichtmann, Portable all-in-one automated microfluidic system (PAMICON) with 3D-printed chip using novel fluid control mechanism. Sci. Rep., 2021. 11(1): p. 1-10.
12. Sun, Y., et al., A valve-based microfluidic device for on-chip single cell treatments. Electrophoresis, 2019. 40(6): p. 961-968.
13. Dong, L. and H. Jiang, Autonomous microfluidics with stimuli-responsive hydrogels. Soft matter, 2007. 3(10): p. 1223-1230.
14. Trasatti, S. and O. A. Petrii, Real surface area measurements in electrochemistry. Pure Appl. Chem., 1991. 63(5): p. 711-734.
15. Shanmugam, S. T., S. Trashin, and K. De Wael, Gold-sputtered microelectrodes with built-in gold reference and counter electrodes for electrochemical DNA detection. Analyst, 2020. 145(23): p. 7646-7653.

1. An aptamer-based sensor comprising an aptamer, the aptamer comprising a nucleic acid sequence sharing at least 50% identity with any one of SEQ ID Nos. 1-24.

2. The aptamer-based sensor according to claim 1 wherein the nucleic acid sequence shares at least 60% identity with any one of SEQ ID Nos. 1-24.

3. The aptamer-based sensor according to claim 1 wherein the nucleic acid sequence shares at least 70% identity with any one of SEQ ID Nos. 1-24.

4. The aptamer-based sensor according to claim 1 wherein the nucleic acid sequence shares at least 80% identity with any one of SEQ ID Nos. 1-24.

5. The aptamer-based sensor according to claim 1 wherein the nucleic acid sequence shares at least 90% identity with any one of SEQ ID Nos. 1-24.

6. The aptamer-based sensor according to claim 1 wherein the nucleic acid sequence shares at least 95% identity with any one of SEQ ID Nos. 1-24.

7. The aptamer-based sensor according to claim 1 wherein the nucleic acid sequence is selected from SEQ ID Nos. 1-24.

8. The aptamer-based sensor according to claim 1 wherein the nucleic acid sequence shares at least 50% identity with SEQ ID No. 22.

9. (canceled)

10. (canceled)

11. The aptamer-based sensor according to claim 1 wherein the nucleic acid sequence shares at least 80% identity with SEQ ID No. 22.

12. (canceled)

13. The aptamer-based sensor according to claim 1 wherein the nucleic acid sequence shares at least 95% identity with SEQ ID No. 22.

14. The aptamer-based sensor according to claim 1 wherein the nucleic acid sequence comprises SEQ ID No. 22.

15. The aptamer-based sensor according to claim 1 wherein the nucleic acid sequence shares at least 50% identity with SEQ ID No. 23.

16. The aptamer-based sensor according to claim 1 wherein the nucleic acid sequence shares at least 95% identity with SEQ ID No. 23.

17. The aptamer-based sensor according to claim 1 wherein the aptamer is bound to a working electrode and the aptamer-based sensor is an electrochemical sensor.

18-35. (canceled)

36. A biosensor comprising a substrate comprising one or more microfluidic channels and an inlet in fluid communication with the one or more microfluidic channels, the one or more microfluidic channels having an assay region, the assay region comprising one or more aptamers that specifically binds to tetrahydrocannabinol or cannabidiol immobilized on an electrode.

37. A method of quantitating tetrahydrocannabinol in a sample, the method comprising:
introducing a sample suspected of containing tetrahydrocannabinol and a redox reagent which binds DNA into the inlet of the biosensor of claim 36;
allowing the sample mixed with redox reagent to flow into the assay region;
allowing any tetrahydrocannabinol or cannabidiol in the sample to bind to the aptamer; and
measuring current generated.

38. A microfluidic device, comprising:
a first layer comprising a counter electrode, a working electrode, and a reference electrode, wherein a surface of said working electrode comprises an aptamer that specifically binds tetrahydrocannabinol or cannabidiol;
a second layer comprising a fluidic channel, said second layer being interfaced with said first layer such that said fluidic channel is in fluid communication with said counter electrode, said working electrode, and said reference electrode, said second layer comprising a fluidic port in fluidic communication with said fluidic channel;
a third layer comprising a chamber for storing a buffer, said third layer further comprising an external port and an internal port in fluid communication with said chamber;
an external seal sealing said external port from an external environment, said external seal being capable of perforation by a pipette tip; and
an internal seal residing between said internal port of said third layer and said fluidic port of said second layer, such that said chamber is brought into fluid communication with said fluidic channel when said internal seal is ruptured;
wherein said third layer is configured such that when the pipette tip perforates said external seal under applied force, a distal portion of said pipette tip is brought into fluid communication with said chamber; and
wherein said internal seal is configured to rupture under applied fluidic pressure, such that injection of a sample from the pipette tip into said chamber results in rupture of said internal seal and flow of the sample and the buffer into said fluidic channel and into fluidic contact with said working electrode, said reference electrode and said counter electrode.

39. The microfluidic device according to claim 38 wherein said external seal is configured such that after perforation of said external seal by the pipette tip, contact between an outer surface of the pipette tip and said external port forms an additional seal under application of an applied force.

40. The microfluidic device according to claim 38, wherein said first layer, said second layer and said third layer are secured via an applied clamping force.

41. A system for providing electrochemical analysis, said system comprising:
a microfluidic device according to any claim 38;
a collection vessel with an opening for collection of a biological fluid sample, said collection vessel further comprising a pipette tip, said pipette tip being capable of perforation of said external seal, and wherein squeezing of said collection vessel causes evacuation of the biological fluid sample into said microfluidic device after perforation of said external seal; and
a potentiostat device connectable to said counter electrode, said working electrode and said reference electrode.


SEQUENCE LISTING (24 sequences)

SEQ ID NO: 1 - DNA, 80 bp, synthetic construct
  source 1..80: mol_type = other DNA
tgtcacatctacactgctcgaaggcgggttttttcacttttaattttcattcattttacctctattcagacagcgttccc

SEQ ID NO: 2 - DNA, 80 bp, synthetic construct
  source 1..80: mol_type = other DNA
tgtcacatctacactgctcgaagacaggctgcttttattcattcatactttcctcgatttaccattcagacagcgttccc

SEQ ID NO: 3 - DNA, 80 bp, synthetic construct
  source 1..80: mol_type = other DNA
tgtcacatctacactgctcgaagcgaatctacaagggctttcttcattctcgttcttccccttattcagacagcgttccc

SEQ ID NO: 4 - DNA, 80 bp, synthetic construct
  source 1..80: mol_type = other DNA
tgtcacatctacactgctcgaagtcttccttttttatcatttttacttactcatgtatttttcattcagacagcgttccc

SEQ ID NO: 5 - DNA, 80 bp, synthetic construct
  source 1..80: mol_type = other DNA
tgtcacatctacactgctcgaaggtctttcgtatttgcattcctctcttcttcatttcgagcaattcagacagcgttccc

SEQ ID NO: 6 - DNA, 37 bp, synthetic construct
  source 1..37: mol_type = other DNA
caagttttttcacttttaattttcattcattttacct

SEQ ID NO: 7 - DNA, 18 bp, synthetic construct
  source 1..18: mol_type = other DNA
caattttttttttttaac

SEQ ID NO: 8 - DNA, 32 bp, synthetic construct
  source 1..32: mol_type = other DNA
tctttcgtatttgcattcctctcttcttcatt

SEQ ID NO: 9 - DNA, 29 bp, synthetic construct
  source 1..29: mol_type = other DNA
aaacaaacaaacatgaatgaaaattaaaa

SEQ ID NO: 10 - DNA, 35 bp, synthetic construct
  source 1..35: mol_type = other DNA
aaacaaacaaacatgaatgaaaaaattaaaaaagt

SEQ ID NO: 11 - DNA, 26 bp, synthetic construct
  source 1..26: mol_type = other DNA
aaacaaacaaacaggaatgcaaatac

SEQ ID NO: 12 - DNA, 26 bp, synthetic construct
  source 1..26: mol_type = other DNA
aaacaaacaaacgaatgcaaaaatac

SEQ ID NO: 13 - DNA, 43 bp, synthetic construct
  source 1..43: mol_type = other DNA
tttgtttgtttgtttgaaaaaattaaaaaaagtaagtacaaac

SEQ ID NO: 14 - DNA, 48 bp, synthetic construct
  source 1..48: mol_type = other DNA
tttgtttgtttgtttaaactgaaaaaaattaaaaaagtaagtacaaac

SEQ ID NO: 15 - DNA, 25 bp, synthetic construct
  source 1..25: mol_type = other DNA
tttgtttgtttgtttcgagcagcat

SEQ ID NO: 16 - DNA, 26 bp, synthetic construct
  source 1..26: mol_type = other DNA
tttgtttgtttgttttcgagcagcat

SEQ ID NO: 17 - DNA, 25 bp, synthetic construct
  source 1..25: mol_type = other DNA
atgctgctcgaaaaattcgagcaat

SEQ ID NO: 18 - DNA, 39 bp, synthetic construct
  source 1..39: mol_type = other DNA
cgggttttttcacttttaattttcattcattttacctct

SEQ ID NO: 19 - DNA, 40 bp, synthetic construct
  source 1..40: mol_type = other DNA
acaggctgcttttattcattcatactttcctcgatttacc

SEQ ID NO: 20 - DNA, 40 bp, synthetic construct
  source 1..40: mol_type = other DNA
cgaatctacaagggctttcttcattctcgttcttcccctt

SEQ ID NO: 21 - DNA, 40 bp, synthetic construct
  source 1..40: mol_type = other DNA
tcttccttttttatcatttttacttactcatgtatttttc

SEQ ID NO: 22 - DNA, 30 bp, synthetic construct
  source 1..30: mol_type = other DNA
tctttcgtatttgcattcctctcttcttca

SEQ ID NO: 23 - DNA, 40 bp, synthetic construct
  source 1..40: mol_type = other DNA
gtctttcgtatttgcattcctctcttcttcatttcgagca

SEQ ID NO: 24 - DNA, 40 bp, synthetic construct
  source 1..40: mol_type = other DNA
tagacagttcatttaactattcttttcactttttctcgtt